(19) United States
(12) Patent Application Publication
(10) Pub. No.: US 2020/0299688 A1
(43) Pub. Date: Sep. 24, 2020

(54) POLYNUCLEOTIDE COMPLEXES HAVING IMPROVED DELIVERY INTO CELLS

(71) Applicant: HER MAJESTY THE QUEEN IN RIGHT OF CANADA, AS REPRESENTED BY THE MINISTER OF AGRICULTURE AND AGRI-FOO, LETHBRIDGE (CA)

(72) Inventors: FRANÇOIS EUDES, LETHBRIDGE (CA); JORDAN PEPPER, LETHBRIDGE (CA)

(21) Appl. No.: 16/087,855

(22) PCT Filed: Mar. 23, 2017
(86) PCT No.: PCT/CA2017/050367

§ 371 Date: Sep. 24, 2018

Related U.S. Application Data
(60) Provisional application No. 62/312,707, filed on Mar. 24, 2016.

(51) Int. Cl. C12N 15/113; A61K 47/24; A61K 47/42

(52) U.S. Cl. CPC C12N 15/113; A61K 47/42; A61K 47/24

(57) ABSTRACT

Compositions including a nanocomplex of a polynucleotide with a cell-penetrating peptide and a quaternary phosphonium salt, such as tetrabutylphosphonium bromide (TBPB), are useful for delivering polynucleotides such as DNA or RNA into cells.

### BACKGROUND

The present application relates to methods of delivery of polynucleotides into cells. More specifically, the present application relates to a method of improving the delivery of polynucleotides into cells mediated by cell-penetrating peptides.

Cell-penetrating peptides (CPPs) are non-viral vectors often derived from the transduction domains of various proteins, and usually consist of 8-30 amino-acids. Often, they are characterized by the presence of a net cationic charge originating from arginine and lysine residues in the sequence of the peptide. Because of their characteristics, which can include the ability to bind nucleotide cargoes through electrostatic interactions, to traverse the cell membrane, and to target specific organelles, these peptides have been used for the delivery of negatively charged polynucleotides into both plant and animal cells. For example, cell-penetrating peptides are described in International Patent Application Publication WO 2008/148223 and organelle targeting polypeptides are described in International Patent Application Publication WO 2013/016810. The delivery of plasmid DNA, double stranded DNA (dsDNA), single stranded DNA (ssDNA) and double stranded RNA (dsRNA) (for example, small interfering RNA (siRNA)) using CPPs has been successful in various plant and animal cell-culture systems.

The success of these systems, however, is based heavily on the physicochemical characteristics of the nanocomplexes formed between the nucleic acids and the CPPs. The principal characteristics that apparently govern the transfection efficiency of these nanocomplexes are size distribution, polydispersity, and zeta-potential. Polydispersity is a measure of the heterogeneity of the sizes of nanoparticles, such that a low polydispersity indicates that the particles in a particular sample have very similar sizes, while a higher polydispersity indicates a sample containing a wider range of particle sizes. Zeta (Q-potential is a measure of the electrostatic repulsion between nanoparticles bearing similar charges. Particles having a higher absolute value of zeta-potential repel each other more strongly, while particles having a lower absolute value of zeta-potential show weaker repulsions, and may have a tendency to attract each other to form larger complexes.

To assess these particular parameters, the standard analysis tool is dynamic light scattering. Dynamic light scattering (DLS) is a photometric technique that analyzes the Rayleigh scattering of monochromatic laser light by colloidal particles in a dispersion medium. This may be used to determine size distribution via the Stokes-Einstein equation or to determine zeta-potential based on electrophoretic mobility of the particles.

The manner of entry of CPPs into the cell has remained a subject of controversy due to conflicting data. However, it has been generally agreed that the process of CPP complex uptake either acts through direct transport, using interactions with the cell membrane phospholipids, or through various forms of endocytosis. In the case of endocytosis, the process is thought to be two-fold; i.e. endosomal formation followed by endosomal release. In order to take advantage of both endocytotic and direct transduction pathways, phase catalysts may improve transfection efficiency.

The cationic components of tetrabutylammonium bromide (TBAB) or tetrabutylphosphonium bromide (TBPB) have been shown to complex with DNA, and are also documented phase-catalysts, binding and moving anions from aqueous hydrophilic environments to non-aqueous hydrophobic environments, possibly encouraging membrane transduction or endosomal release. As well, polymers with tertiary ammonium and phosphonium groups have been utilized for delivery of nucleic acid cargoes into animal cells, and phosphonium salts have been shown to have mitochondrial targeting properties. Polyphosphonium polymers have shown promise as low cytotoxicity transfection agents superior to their ammonium analogues, able to effectively complex nucleic acids at 1:1 molar charge ratios.

It has been proposed that this highly efficient complexation is due to the higher localization of cationic charge on the phosphorus atom of the quaternary phosphonium versus the quaternary ammonium nitrogen atom. Tertiary phosphonium and ammonium cations of various types have been shown to interact strongly with DNA, and to show similar interactions with dsRNA. However previous work used phenyl substituted phosphonium cations, and intercalative and electrostatic interactions with similar organic groups to form hydrophobic interactions were observed.

Most the work performed with CPPs has focused on delivery in animal cells, mammalian cells in particular, which have shown high efficiency delivery of a number of cargoes conjugated or non-covalently bound to CPPs. The same cannot be said for plant cells, due to factors inhibiting uptake, such as the cell wall, differing rates of macropinocytosis and differences in membrane physicochemistry. One particular difference not previously investigated is the composition of plant tissue culture media and its effect on complex formation and stability. Because of the overriding dominance of animal cell culture systems as research platforms for CPP investigation, the relevant media investigated has frequently been PBS (phosphate-buffered saline) or serum based media. As a result very little information gathered from other complex formation studies is applicable to plant tissue culture systems which use high sugar media.

Therefore, it is desirable to provide alternative compositions and methods, and improvements thereof, for CPP-mediated delivery of polynucleotides to cells, including but not limited to plant cells.

### SUMMARY

In one aspect, the present invention provides a composition for delivering a polynucleotide into a cell, the composition comprising a cell-penetrating peptide, the polynucleotide and a quaternary phosphonium salt. In at least one embodiment, the quaternary phosphonium salt is tetrabutylphosphonium bromide. In at least one embodiment, the polynucleotide is a DNA molecule. In at least one embodiment, the polynucleotide is a double-stranded RNA molecule. In at least one embodiment, the cell is a plant cell.

Another aspect of the present invention provides a composition for transfecting a cell, the composition comprising a cell-penetrating peptide, a polynucleotide and a quaternary phosphonium salt. In at least one embodiment, the quaternary phosphonium salt is tetrabutylphosphonium bromide. In at least one embodiment, the polynucleotide is a DNA molecule. In at least one embodiment, the cell is a plant cell.

A further aspect of the present invention provides a method of delivering a polynucleotide into a cell, comprising exposing the cell to a composition as described herein.

In another aspect, the present invention provides a method of transfecting a cell, comprising exposing the cell to a composition as described herein.

An additional aspect of the present invention provides a method of reducing or preventing expression of a gene in a cell, the method comprising exposing the cell to a composition as described herein, wherein the polynucleotide is a double stranded RNA (dsRNA) molecule specific to the gene.

### BRIEF DESCRIPTION OF DRAWINGS

Further features of the present invention will become apparent from the following written description and the accompanying figures, in which:

FIG. 1 is a series of graphs showing the effect of ionic strength (I_{c}) on hydrodynamic diameter (D_{H}) and polydispersity index (PDI) (means±standard deviation; n=3) of Tat₂-dsRNA nanocomplexes with various cationic to anionic (N:P) molar charge ratios (1:1 (panels a and b), 4:1 (panels c and d) and 8:1 (panels e and f)) in maltose-mannitol (MM) solutions in the absence (control) or presence of tetrabutylammonium bromide (TBAB) or tetrabutylphosphonium bromide (TBPB);

FIG. 2 is a series of graphs showing the effect of the number of base pairs on hydrodynamic diameter (D_{H}) and polydispersity index (PDI) (means±standard deviation; n=3) of Tat₂-dsRNA or Tat₂-DNA nanocomplexes having a cationic to anionic (N:P) molar charge ratio of 8:1 in the absence (panels a and c) or presence (panels b and d) of tetrabutylphosphonium bromide (TBPB), at various values of ionic strength;

FIG. 3 is a graph plotting hydrodynamic volume (V_{H}) of Tat₂-nucleic acid nanocomplexes of minimal volume vs. approximate length of nucleic acid;

FIG. 4 is a bar graph showing zeta-potential of circular plasmid and dsRNA samples alone, or mixed with Tat₂ at N:P ratios of 1:1, 4:1 and 8:1, in the absence or presence of tetrabutylphosphonium bromide (TBPB);

FIG. 5 is a bar graph showing relative expression of the phytoene desaturase gene (pds) from seedling Triticale (Sunray) leaves converted from ΔΔCt values, normalized against expression of the adp-rfgene. Data displayed are means±standard error (n=8). (*) and (**) indicate a significance of p<0.05 and p<0.0001, respectively, between the designated treatment and the control (solvent only), based on Student's t-test;

FIG. 6 is a photograph of Colorado potato beetles (Leptinotarsa decemlineata) scored as surviving, lethargic or dead;

FIG. 7 is a graph showing the loss of vitality index (LoVI) and the relative expression of the p5cdh gene in Colorado potato beetles treated with varying dosages of p5cdh siRNA:Tat₂:TBPB at a ratio by weight of 1:4:100;

FIG. 8 is a bar graph showing loss of vitality index (LoVI) and relative expression of the p5cdh and arf1 genes in Colorado potato beetles treated with a dose of 2.5 μg p5cdh siRNA:10 μg Tat₂:250 μg TBPB, or with water alone (control), TBPB alone, siRNA alone or a complex of Tat₂: siRNA in the absence of TBPB; and

FIG. 9 is a photograph of an agarose gel showing the relative sizes of RNA fragments from lysates from Colorado potato beetles treated as described for FIG. 8 (A: water alone; B: TBPB alone; C: siRNA alone; D: Tat₂:siRNA; E: Tat₂:siRNA:TBPB).

### DETAILED DESCRIPTION

In one aspect, the present invention provides a composition for delivering a polynucleotide into a cell or for transfecting the cell with the polynucleotide. The composition comprises the polynucleotide, a cell-penetrating peptide and a quaternary phosphonium salt. In at least one embodiment, the quaternary phosphonium salt is tetrabutylphosphonium bromide. In at least one embodiment, the cell-penetrating peptide has a net cationic charge. In at least one embodiment the cell-penetrating peptide has a sequence of from about 8 to about 30 amino acid residues. In at least one embodiment, the cell-penetrating peptide is Tat₂ (RKKRRQRRRRKKRRQRRR, SEQ ID NO:1). Other cell-penetrating peptides known in the art, including but not limited to cell-penetrating peptides described in International Patent Application Publication WO 2008/148223 and organelle targeting polypeptides described in International Patent Application Publication WO 2013/016810, are also contemplated as useful for the present invention.

In at least one embodiment, the polynucleotide is selected from DNA and RNA. In at least one embodiment, the polynucleotide is selected from DNA and double-stranded RNA. In at least one embodiment, the polynucleotide is DNA. In at least one embodiment, the polynucleotide is double-stranded RNA. In at least one embodiment, the polynucleotide is double-stranded RNA having about 20 to about 30 base pairs. In at least one embodiment, the polynucleotide is a small interfering RNA (siRNA).

In at least one embodiment, the composition comprises an amount of the cell-penetrating peptide and an amount of the polynucleotide such that the molar charge ratio of the cationic charge of the cell-penetrating peptide to the anionic charge of the phosphate backbone of the polynucleotide is in a range from about 1:1 to about 8:1. In at least one embodiment, the composition comprises the quaternary phosphonium salt in an amount such that the molar charge ratio of the cationic charge of the phosphonium salt to the anionic charge of the phosphate backbone of the polynucleotide is about 100:1. In at least one embodiment, the composition comprises the nucleotide, the cell penetrating peptide and the quaternary phosphonium salt in a weight ratio of 1:4:100. The skilled person would be readily able, in light of the teachings herein and using only routine experimental procedures well known in the art, to determine other relative amounts of the cell-penetrating peptide, the polynucleotide and the quaternary phosphonium salt which are useful or optimal under various conditions.

In at least one embodiment, the composition comprises an acceptable carrier. In at least one embodiment, the carrier is water. In at least one embodiment, the carrier is a mannitol-maltose solution. In at least one embodiment, the carrier further comprises a salt. In at least one embodiment, the salt is calcium chloride. In at least one embodiment, the salt is present in an amount sufficient to provide an ionic strength of up to 1.0 M. In at least one embodiment, the salt is present in an amount sufficient to provide an ionic strength of 0.10 M, 0.20 M, 0.40 M, 0.60M, 0.80M or 1.0 M. The skilled person would be readily able, in light of the teachings herein and routine experimental procedures well known in the art, to identify, select and prepare other suitable carriers.

In at least one embodiment, the cell is a plant cell. In at least one embodiment, the cell is an insect cell. In at least one embodiment, the cell is an animal cell.

The present inventors have surprisingly found that the present composition can, in at least one embodiment, have one or more advantageous properties. Specifically, in at least one embodiment, the nanocomplex formed between the polynucleotide, the cell-penetrating peptide and the quaternary phosphonium salt in the present composition can have one or more of a reduced size (as indicated, for example, by a reduced hydrodynamic diameter), an increased uniformity (as indicated, for example, by a reduced polydispersity index) and/or an increased repulsion for other particles of like charge (as indicated, for example, by an increased absolute value of zeta-potential).

Without being bound by theory, it is contemplated that the addition of quaternary phosphonium salts to a mixture of a cell-penetrating polypeptide (CPP) and a polynucleotide can act as a chaotropic which is effective at low concentrations to break apart larger aggregates into smaller particles. Cell-penetrating polypeptides (CPP) generally have a net positive charge due to the presence of lysine and/or arginine residues, and polynucleotides generally have a net negative charge due to the presence of backbone phosphate residues. In addition, quaternary phosphonium salts include positively-charged phosphonium ions. It is thought that complexation of the positively-charged phosphonium ions with the CPP and polynucleotide may allow complete binding to form nanocomplex particles at a lower charge ratio load of CPP molecules to polynucleotide molecules. In addition, inter-particle repulsion may be increased, providing a smaller particle size and/or a more homogeneous particle size distribution.

Furthermore, it is thought that increasing the ionic strength of the mixture may help stabilize the nanocomplex particles, possibly through electrostatic screening, and thus increase the uniformity of the size of the particles. In addition, it is thought that the use of a medium high in maltose and mannitol may also contribute to the apparent chaotropic activity of TBPB.

### Definitions

As used herein, the term “nanocomplex” is intended to mean a complex formed by mixing a cell-penetrating polypeptide (CPP) and a polynucleotide. A nanocomplex can include one or more other components, including but not limited to particles, molecules or ions which may carry a full or a partial charge. For example, a nanocomplex may also include one or more quaternary phosphonium ions.

As used herein interchangeably, the term “cell-penetrating polypeptide” or “CPP” is intended to mean a polypeptide which is capable of binding a polynucleotide such that the complex formed can traverse the cell membrane of a cell.

As used herein, the term “polynucleotide” is intended to mean a polymer formed from ribonucleotides (polyribonucleotides or ribonucleic acids (RNA)), or deoxyribonucleotides (polydeoxyribonucleotides or deoxyribonucleic acids (DNA)), or RNA-DNA hybrids, as known in the art. The polynucleotides can have any length from 2 nucleotides or base pairs to millions or billions or more of nucleotides or base pairs. Polynucleotides can be double-stranded or single-stranded, circular or linear, or naturally occurring or partially or completely synthetic.

As used herein interchangeably, the term “siRNA”, or “small interfering RNA”, is intended to mean a double stranded RNA molecule having a sequence of 20 to 30 nucleotides which is active, or capable of being active, to mediate the degradation of specific messenger RNA (mRNA) sequences by the RNA interference pathway, as is well known in the art. siRNA molecules are therefore capable of being active to at least temporarily silence the expression of specific genes.

As used herein, the term “quaternary phosphonium salt” is intended to mean a salt including a quaternary phosphonium cation and an anion. As used herein interchangeably, the term “quaternary phosphonium cation” or “quaternary phosphonium ion” is intended to mean a positively charged ion of general formula R₄P⁺, where R in each instance independently is a hydrocarbyl group, including but not limited to an alkyl group or an aryl group. Suitable alkyl groups are known in the art, and include but are not limited to C₁₋₆ alkyl groups which may be linear or branched and C₁₋₆ cycloalkyl groups. Suitable aryl groups include but are not limited to phenyl groups, as known in the art. Suitable alkyl, cycloalkyl and aryl groups can optionally be substituted with one or more C₁₋₆ alkyl groups.

As used herein interchangeably, the terms “hydrodynamic diameter” and “D_{H}” are intended to mean the diameter of a hypothetical hard sphere which has the same diffusion behavior as a particle being studied. Hydrodynamic diameter is determined by measuring the diffusion behavior of a particle in solution, and is therefore indicative of the apparent size of the solvated or hydrated particle.

As used herein, the term “zeta-potential” is intended to refer to the electrostatic potential difference between the stationary layer of fluid around a particle dispersed in a dispersion medium and a point in the bulk dispersion medium away from the slipping plane or interface between the particle and the medium. Zeta potential is a measure of the electrostatic repulsion between nanoparticles bearing similar charges which are dispersed in a medium.

As used herein, the term “polydispersity index” or “PDI” is intended to mean a parameter calculated from data obtained from a dynamic light scattering experiment carried out on a sample containing sub-micrometer sized particles dispersed in a medium. The value of the polydispersity index indicates the degree of heterogeneity of the sizes of the particles in the sample subjected to the experiment. Calculation of polydispersity index can be carried out as described in ISO standard 22412:2017, revising ISO standards 22412:2008 and 13321:1996.

As used herein, the terms “about” or “approximately” as applied to a numerical value or range of values are intended to mean that the recited values can vary within an acceptable degree of error for the quantity measured given the nature or precision of the measurements, such that the variation is considered in the art as equivalent to the recited values and provides the same function or result. For example, the degree of error can be indicated by the number of significant figures provided for the measurement, as is understood in the art, and includes but is not limited to a variation of ±1 in the most precise significant figure reported for the measurement. Typical exemplary degrees of error are within 20 percent (%), preferably within 10%, and more preferably within 5% of a given value or range of values. Alternatively, and particularly in biological systems, the terms “about” and “approximately” can mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

As used herein, the term “substantially” refers to the complete or nearly complete extent or degree of an action, characteristic, property, state, structure, item, or result. For example, two entities or properties which are “substantially” similar would mean that the entities or properties are either completely identical or are similar within an acceptable tolerance. The exact allowable degree of deviation from absolute completeness may in some cases depend on the specific context. However, generally speaking the nearness of completion will be so as to have the same overall result as if absolute and total completion were obtained.

The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

### EXAMPLES

Other features of the present invention will become apparent from the following non-limiting examples which illustrate, by way of example, the principles of the invention.

### Example 1: Nucleic Acid Preparations

Final concentrations of all nucleic acids were determined using a NanoDrop 8000 UV-Vis spectrophotometer (Thermo Scientific).

Linear Double Stranded DNA:

A 1 kb ladder (Invitrogen) prepared according to the manufacturer's instructions was used as the source of linear double stranded DNA (dsDNA) fragments. The ladder was separated by electrophoresis on a 1.0% agarose gel stained with Gel Red (0.5×) for 1 hour in 1× Tris-Acetate-EDTA (TAE) buffer at 90 V. Bands containing linear dsDNA fragments at 0.5, 1 and 3 kb were visualized using an ultraviolet light and excised from the gel using a scalpel. The DNA fragments were then purified from the individual agarose gel bands using the Qiagen gel extraction kit, following the manufacturer's instructions and eluting all final fragments from columns using ultrapure water (Sigma-Aldrich). Isolated DNA stock solutions were diluted to a concentration of 20 ng/μL.

Circular Plasmid DNA:

The circular pMS plasmid (5556 bp) was cloned using E. coli strain DH5α. E. coli containing pMS was taken from a glycerol stock, cultured overnight in 200 mL of LB (lysogeny broth or Luria-Bertani) medium and purified from cells using a plasmid maxiprep kit according to the manufacturer's instructions (Sigma-Aldrich). Plasmid was eluted from the included columns using ultrapure water (Sigma-Aldrich) and diluted to a stock concentration of 20 ng/μL.

Linear Double Stranded RNA:

A 21-base pair (21mer) dsRNA was obtained from IDT (Integrated DNA Technologies), having the sequence 5′-CAUGGAGACGCCGUCGUU-3′ (SEQ ID NO: 2) and complementary strand 5′-CGAGGACGGCGUCUCCAUGUU-3′ (SEQ ID NO: 3), and producing a duplex with double U overhangs and a molecular weight of 13368.1 Da. The lyophilized product was dissolved in Ultrapure Water and subsequently diluted to a stock concentration of 2.0 μM (26.7 ng/μL).

### Example 2: Preparation of Nanocomplex Samples

Sample preparation and analysis methods were based on those reported in Jafari et al., (2014) “Serum stability and physicochemical characterization of a novel amphipathic peptide C6M1 for siRNA delivery”, PLoS One, 9(5), e97797. Individual samples were prepared in a polymerase chain reaction (PCR) well plate. Maltose-mannitol (MM, 90 mg/mL maltose, 9 mg/mL mannitol) solutions with varying concentrations of CaCl₂ (0.00 mM, 78.43 mM, 156.86 mM, 235.29 mM, 313.73 mM and 392.16 mM) were prepared in double distilled water (ddH₂O) at pH 7.0 and filtered twice through 0.2 μm cellulose syringe filters (VWR) using a 5 mL syringe. From these stock solutions, 17 μL aliquots for each CaCl₂ concentration were added into well plates, followed by 1 μL of stock dsRNA or DNA solution. Finally, 1 μL of Tat₂ peptide was added in an amount calculated to provide Tat₂:dsRNA cationic:anionic (+/−) molar charge ratios (N⁺:backbone phosphate or N:P) of 1:1, 4:1 and 8:1 or a Tat₂:DNA+/−molar charge ratio (N:P) of 8:1. A further 1 μL of MM solution containing no CaCl₂ was added to bring the final volume to 20 μL. The reaction mixtures were allowed to incubate for 15 minutes. In the case of the addition of tetrabutylammonium bromide (TBAB, Sigma) and tetrabutylphosphonium bromide (TBPB, Sigma), 1 μL of 8.4 nmol/μL TBAB or TBPB dissolved in MM was added after 10 minutes of incubation time, instead of the further 1 μL of MM solution containing no CaCl₂ and the mixture was incubated for a further 5 minutes. This provided a +/−100:1 molar charge ratio of N⁺ or P⁺ to backbone phosphates. These preparations resulted in 20 μL samples with final CaCl₂ concentrations of 0.0, 66.6, 133.3, 200.0, 266.6 and 333.3 mM, corresponding to ionic strengths (I_{c}) of 0.0, 0.2, 0.4, 0.6, 0.8 and 1.0 M respectively. The final concentrations of TBAB and TBPB were 420 μM, contributing little to the ionic strength of the solution. Only mixtures having the 8:1 charge ratio and TBPB were tested with DNA samples.

### Example 3: Size Analysis

Size Measurement:

Size analysis was carried out immediately after incubation in a low volume quartz sizing cuvette (ZEN 2112) wetted with 200 μL MM solution. The entire sample was loaded into the cuvette and the size of the nanoparticles was measured by dynamic light scattering (DLS) on a Zetasizer Nano ZS (Malvern) with a 633 nm laser at 173° backscatter. Data was analyzed using the CONTIN algorithm in the Zetasizer software v.7.02 (Malvern). Three repeat measurements were made of each sample, and means and standard deviations of both the primary particle size distribution peak, and polydispersity indices (PDI) were calculated and plotted. Size is measured as hydrodynamic diameter (D_{H}), and is therefore reflective of the apparent size of the particle in solution, and is best applied to comparing relative sizes of particles under different conditions.

Tat₂ Complexation with dsRNA:

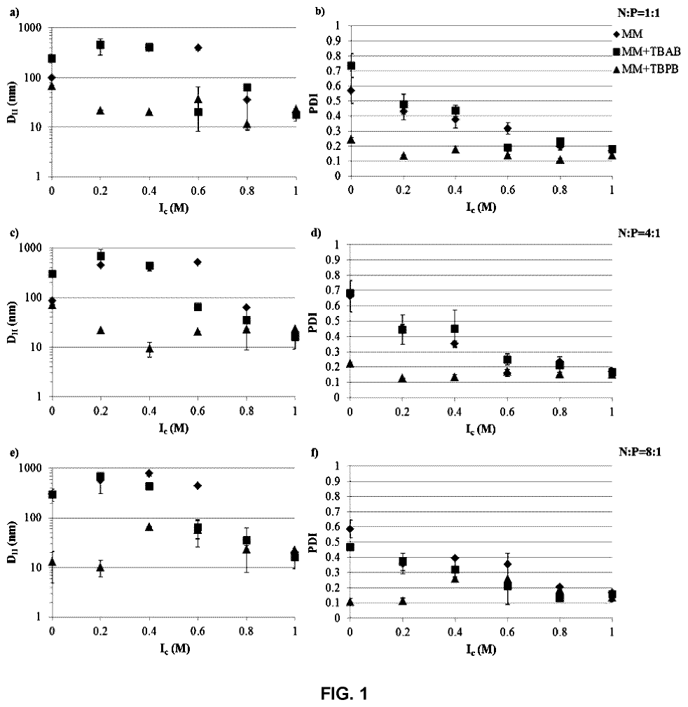

Hydrodynamic diameters (D_{H}) and polydispersity indices (PDI) of Tat₂:dsRNA nanocomplexes of varying cationic:anionic molar charge ratios and ionic strengths, in the presence or absence of TBAB or TBPB are shown in FIG. 1. As seen from FIG. 1, Tat₂ complexation with 21-mer dsRNA showed significant reduction in particle size as indicated by D_{H} (panels a, c and e) and PDI (panels b, d and f) in the presence of TBPB at lower ionic strengths. It can be seen that at all charge ratios of Tat₂:dsRNA, the presence of TBPB at a +/−100:1 molar charge ratio decreased particle size but showed the largest effect at the 8:1 Tat₂:dsRNA charge ratio, producing nanocomplexes with a D_{H} of about 10 nm even in the absence of CaCl₂ (FIG. 1, panel e). In contrast, the nanocomplexes formed from the 8:1 charge ratio of Tat₂:dsRNA had a D_{H} of about 300 nm in the absence of TBPB. In addition, nanocomplexes produced in the presence of TBPB from the mixture having an 8:1 Tat₂:dsRNA charge ratio also showed the lowest value of PDI (about 0.1), indicating that the particles are also highly homogeneous in size. As ionic strength increased above 0.6 M, the effect of TBPB on particle size and PDI became indistinguishable from the effect of CaCl₂ alone. The presence of TBAB only had a noticeable effect on particle size and PDI at an ionic strength of 0.6 M.

Tat₂ Complexation with DNA:

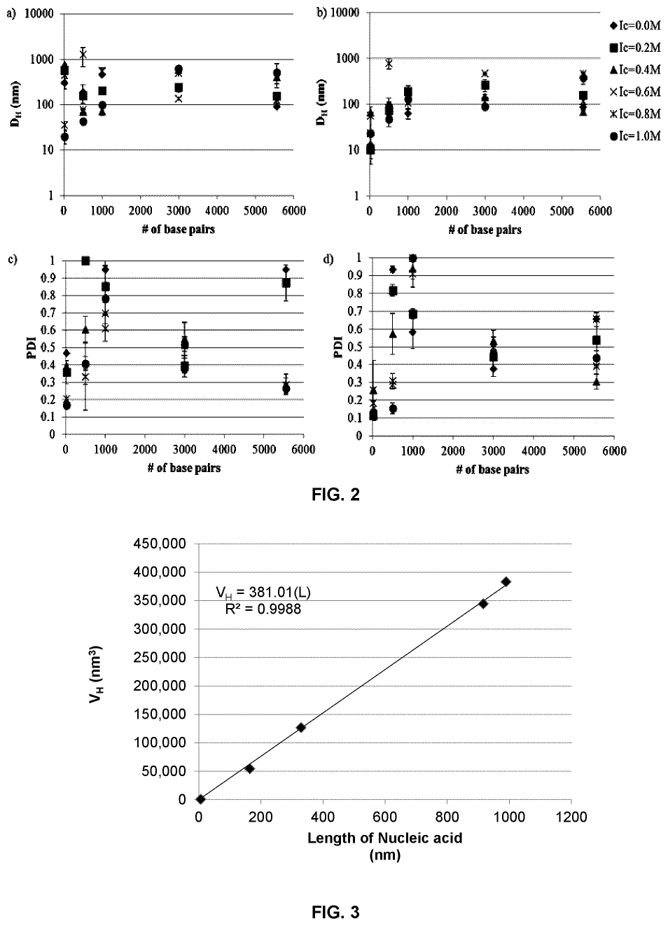

Hydrodynamic diameters (D_{H}) and polydispersity indices (PDI) of Tat₂:DNA nanocomplexes having a cationic:anionic molar charge ratio of 8:1 and varying numbers of base pairs at varying ionic strengths and in the absence (panels a and c) or presence (panels b and d) of TBPB are shown in FIG. 2. Data for the Tat₂:dsRNA nanocomplexes discussed above having a cationic:anionic molar charge ratio of 8:1 and no more than 21 base pairs are included for comparison. The addition of TBPB to Tat₂:DNA nanocomplexes showed a smaller effect on D_{H} and PDI than that observed with the dsRNA oligomer.

In the case of 0.5 kb DNA, TBPB had a weak effect on PDI at low ionic strength, but reduced particle size by about 50%, from 189.3 nm to 96.0 nm. This effect became more pronounced with an increase in ionic strength (I_{c}), however, at ionic strength of 0.8 M, the size increased to 811 nm, with reduction to a minimum in size at an I_{c} of 1.0 M of 46.95 nm. The PDI was found to generally decrease with increasing I_{c} with a reduction to 0.155 at an I_{c} value of 1.0 M. It is noted that the D_{H} of the 0.5 kb linear DNA sample at 0.8 M I_{c} CaCl₂ in the presence of TBPB measured about 800 nm, and was larger than that of the non-TBPB formulation (about 80 nm), possibly due to the complex structural dynamics observed for long linear DNA, which has more degrees of conformational freedom.

In the case of the 1 kb DNA fragment, the largest difference in size and PDI between measurements in the absence and presence of TBPB can be seen at 0.0 M ionic strength, where size was at a minimum (62.6 nm) and PDI was 0.538 in the presence of TBPB. In comparison, the minimum size in the absence of TBPB (72.6 nm) was reached at 0.4 M ionic strength. The PDI was high at all ionic strengths higher than 0.0 M, both in the presence and absence of TBPB.

For the 3 kb DNA fragment, the PDI was similar in the presence and absence of TBPB. However, size was generally reduced to 90-120 nm in the presence of TBPB under all ionic strengths except for I_{c}=0.2 M where the size of the particles was similar in the presence and absence of TBPB (about 250 nm) and I_{c}=0.6 M, where the size was significantly smaller in the absence of TBPB (135.9 nm) than in its presence (461.5 nm).

For the circular plasmid (5.5 kb), the sizes of the nanocomplex particles were similar in the presence and absence of TBPB, up to 0.2 M ionic strength and at 0.6 M. PDI decreased significantly in the presence of TBPB at low ionic strength (a reduction of 0.3), but the effect decreased as ionic strength increased above 0.4 M.

In general, the effect of TBPB to reduce the size and PDI of nanocomplexes was stronger for smaller polynucleotides and at lower ionic strengths. However, for each polynucleotide studied, the minimal size was observed in the presence of TBPB.

Advanced Size Analysis:

Formulations were identified which resulted in minimally sized nanocomplexes for each nucleic acid studied. In each case, the identified formulation contained TBPB. Approximate lengths of each nucleic acid were calculated based on an assumed length of 0.33 nm per bp. For each of these formulations, hydrodynamic diameters (mean measured sizes, D_{H}) were used to calculate hydrodynamic volumes (V_{H}) under the assumption of a near spherical shape for all nanocomplexes. The data is shown in Table 1.

TABLE 1
Minimally Sized Nanocomplexes and Formulations

| | I_{c} | Minimal | Calculated | Goodness of |
|---|---|---|---|---|
| Nucleic acid: number of | CaCl₂ | D_{H} | V_{H} | Regression fit |
| nucleotides/length (nm) | (M) | (nm) | (nm³) | (% of slope) |
| dsRNA (21/6.93) | 0.2 | 10.29 | 572.0 | 21 |
| Linear dsDNA (500/165) | 1.0 | 46.95 | 5.419 · 10⁴ | 86 |
| Linear dsDNA (1000/330) | 0.0 | 62.30 | 1.266 · 10⁵ | 100 |
| Circular plasmid | 1.0 | 86.96 | 3.443 · 10⁵ | 99 |
| (5.556/916.7) | | | | |
| Linear dsDNA (3000/990) | 0.4 | 90.10 | 3.830 · 10⁵ | 101 |

FIG. 3 shows a plot of V_{H} as a function of the approximate length of the nucleic acid. In the case of the circular plasmid, the length was halved to account for supercoiling and circular shape. Linear regression was used to determine a mathematical relationship between nucleic acid length and nanocomplex particle hydrodynamic volume, and the slope was used to determine the goodness of fit of each individual data point. As seen from FIG. 3, a robust linear regression (R²=0.9988) was observed for the equation:

V_{H}=381.01(L)

where L is the length of the nucleic acid. The linear regression was forced through zero to better reflect the fact that a length of 0 nm would result in a nanocomplex of 0 nm³.

The slope of the linear regression fit based on the equation V_{H}=381.01(L) was compared to the quotient of V_{H} over L, to determine how well the calculated values of V_{H} correspond to the spherical approximation. The poorest fit was found for 21-mer dsRNA at 21% similarity to the slope when the calculated length and volume were used (i.e. V_{H}/L=572.0/6.93=82.5, which is only 21% of the determined slope of 380.01). In other words, the hydrodynamic volume (572.0 nm³) of the nanocomplex formed from the 21-mer dsRNA in the presence of TBPB, as determined from the observed minimal hydrodynamic diameter, is only 21% of that expected (381.01×6.93, or 2640.40 nm³) from the trend observed with the larger (500 to 5,556 base pairs) polynucleotides. Thus, it appears that the effect of TBPB to reduce the size of the nanocomplexes is stronger for polynucleotides having fewer base pairs.

### Example 4: Zeta-Potential Analysis

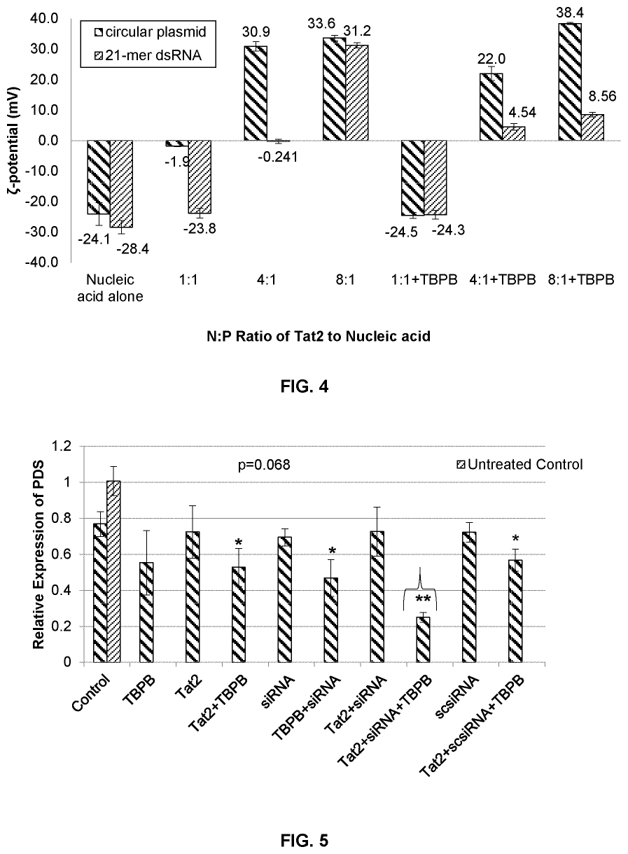

Zeta (Q-potentials were analyzed for circular plasmid DNA, and for 21-mer dsRNA in formulations prepared as described in Example 2 but with a five-fold increase in the concentration of nucleic acid and Tat₂ (5 ng/μL of nucleic acid). Measurements were taken for formulations including Tat₂ and having a 1:1, 4:1 and 8:1 N:P ratio for each polynucleotide, both in the presence and absence of TBPB (100:1+/−molar ratio), as well as for the polynucleotide alone. Zeta-potential values (mV) of samples were determined using a capillary zeta-cuvette with gold electrodes in a Zetasizer-Nano (Malvern). The samples were prepared to a volume of 700 μL to fill the cuvette. Data displayed represent means±standard deviation (s.d.) calculated from triplicate measurements of a single sample. The results are shown in FIG. 4.

As seen in FIG. 4, for the circular plasmid DNA, addition of Tat₂ at an N:P ratio of 1:1 caused a significant change in the zeta-potential from −24.1 to −1.9, while further addition of TBPB caused a further significant change in the zeta-potential to −24.5. At an N:P ratio of 4:1, addition of TBPB to the Tat₂:DNA nanocomplex changed the zeta potential from +30.9 to +22.0, and at an N:P ratio of 8:1, addition of TBPB to the Tat₂:DNA nanocomplex changed the zeta potential from +33.6 to +38.4.

For the double stranded RNA, addition of Tat₂ at an N:P ratio of 1:1 changed the zeta-potential from −28.4 to −23.8, and further addition of TBPB changed the zeta potential minimally to −24.3. At an N:P ratio of 4:1, addition of TBPB to the Tat₂:dsRNA nanocomplex changed the zeta potential from −0.241 to +4.54, and at an N:P ratio of 8:1, addition of TBPB to the Tat₂:dsRNA nanocomplex changed the zeta potential from +31.2 to +8.56.

Thus, for Tat₂-polynucleotide nanocomplexes with higher cationic:anionic ratios, the presence of TBPB has a greater effect on reducing zeta-potential for the smaller dsRNA than for the larger circular plasmid.

### Example 5: Delivery of siRNA in Young Triticale Leaf Tissue

Treatment of Plant Material and Sample Collection:

Seeds of Triticosecale sp. Whittmack cv Sunray, were planted in 8×4 Rootrainers™ and allowed to grow in a growth cabinet at 12-15° C., and a photoperiod of 19 hr/day at an intensity of 300 μE/m²/s. At 13 days post planting, at the two leaf stage, plants were inoculated using a 1 mL sterile syringe (VWR) to inject a formulation of one of the following in either double distilled water (ddH₂O) or maltose-mannitol (MM) solution: solution only (Control), TBPB only, siRNA only, Tat₂ only, TBPB+Tat₂, TBPB+siRNA, siRNA+Tat₂, Tat₂+siRNA+TBPB, scsiRNA (scrambled siRNA), and Tat₂+scsiRNA+TBPB. A total of eighteen treatments were performed in a minimum of four replicates, where a single repetition consisted of eighteen individual plants. A total of 250 pmol of siRNA in 100 μL was injected in all siRNA treatments, with TBPB and Tat₂ being added proportional to the siRNA at the 8:1 N:P ratio of Tat₂ to siRNA and TBPB at the +/−100:1 ratio of phosphonium ions to phosphates. The injections were directed towards the base of the youngest leaf on the bottom side. Plants were placed back into their prior growth conditions until sample collection 24 hours later.

The siRNA duplex (IDT) was targeted to the phytoene desaturase endogenous gene (pds) and was designed using pssRNAit online tool provided by the Zhao Bioinformatics Laboratory based on a Chinese spring wheat pds mRNA sequence (GenBank accession number FJ517553.1) with the sequence 5′-CAUGUUGUGAAGACACCCGAG-3′ (sense, SEQ ID NO:4) and 5′-UCGGUGUCUUCACAACAUGGU-3′ (anti-sense, SEQ ID NO:5).

To generate the scsiRNA duplex (5′-AGCCGGUACGAAUAGTGAGUC-3′ (SEQ ID NO:6) and 3′-UCGGCCAUGCUUAUCACUCAG-5′ (SEQ ID NO:7)), the siRNA sense sequence was scrambled using the Genscript® online sequence scrambler; with rice being used as a reference genome. Both strands of the resultant sequence were tested for potential off-target sequence alignment with the Ensembl Plants database (Triticum aestivum and Hordeum vulgare genomes), as well as the general NCBI database using BLASTn.

RNA Extraction, cDNA Synthesis, and RT-qPCR Analysis:

The tip of the inoculated leaves (3-4 cm length by 1 cm width) was cut using scissors and placed into screwcap “tough tubes” (VWR) with 3 stainless steel beads (3 mm width) and immediately frozen in liquid nitrogen and stored at −80° C. To extract RNA, a NucleoMag™ RNA 96 well plate (Macherey-Nagel) extraction kit was used, according to the manufacturer's instructions with automation of extraction on a BioSprint 96 well plate robot (Qiagen). Alternatively, RNA was extracted using an RNeasy™ Plant mini kit (Qiagen). Purified RNA was quantified using UV-vis spectroscopy on a NanoDrop™ 8000 (ThermoFisher Scientific). cDNA was synthesized using a Superscript® VILO cDNA synthesis kit (ThermoFisher Scientific) according the manufacturer's instructions with a total of 1.2 μg of RNA template per 20 μL reaction. RT-qPCR analysis was performed using SYBR™-Green master mix (Life Technologies) with 40 ng of template cDNA per PCR reaction in 96 well plates. Each treatment and biological replicate was evaluated in three technical PCR replicates, with primers (500 nM forward and reverse) designed to amplify the target cDNA of phytoene desaturase (pds) and of three reference genes; ADP-ribosylation factor (adp-rf), cell division control protein (cdc) and RNase L inhibitor protein (rl1). Primer sequences are provided in Table 2.

TABLE 2
Primer-set Sequences for RT-qPCR

| | Forward Primer Sequence | Reverse Primer Sequence | |
|---|---|---|---|
| Gene | (5′-3′) | (5′-3′) | Source |
| pds | AAAGCAGGGTGTTCCTGAT | CATGGATAACTCGTCAGGGTTTA | Geneious software |
| | (SEQ ID NO: 8) | (SEQ ID NO: 9) | alignment with NCBI |
| | | | FJ517553.1 |
| adp-rf | TCTCATGGTTGGTCTCGATG | GGATGGTGGTGACGATCTCT | (Gimenez, M. J.et al |
| | (SEQ ID NO: 10) | (SEQ ID NO: 11) | (2011). Planta, 233, |
| cdc | CAGCTGCTGACTGAGATGGA | ATGTCTGGCCTGTTGGTAGC | 163-173) |
| | (SEQ ID NO: 12) | (SEQ ID NO: 13) | |
| rl1 | TTGAGCAACTCATGGACCAG | GCTTTCCAAGGCACAAACAT | |
| | (SEQ ID NO: 14) | (SEQ ID NO: 15) | |

Thermocycling conditions were the following; 95° C. for 15 min, and 40 cycles of 15 s of 95° C., 30 s of 60° C. and 30 s of 72° C., followed by a melting temperature series to evaluate amplicon content; 15 s at 95° C., 15 s at 60° C. and 15 s 95° C. Reference genes were analyzed for stability by global averages of Ct-values. Table 3 shows the raw Ct values for 42 of the samples collected.

TABLE 3
Ct value Analysis of Target (pds) and Reference Genes

| | | Target Gene | | Reference Genes | | | | | |
|---|---|---|---|---|---|---|---|---|---|
| | | pds | | adp-rf | | cdc | | rl1 | |
| Treatment | | ddH₂O | MM¹ | ddH₂O | MM¹ | ddH₂O | MM¹ | ddH₂O | MM¹ |
| Control | R1 | 25.97506 | 25.27929 | 23.69714 | 23.64619 | 24.89149 | 24.59786 | 27.55565 | 26.8921 |
| | R2 | 24.09459 | 22.97824 | 22.32155 | 22.68338 | 22.50624 | 22.97363 | 25.32632 | 25.50647 |
| | R3 | 24.89621 | 25.0647 | 23.08394 | 24.07086 | 23.60398 | 25.33332 | 26.07088 | 27.85261 |
| TBPB | R1 | 23.8075 | 26.51612 | 23.5131 | 23.30975 | 19.80063 | 24.19953 | 24.34309 | 28.25299 |
| | R2 | 25.19291 | 23.16497 | 22.32155 | 22.94547 | 21.94078 | 21.53990 | 25.55262 | 25.74529 |
| | R3 | 24.78741 | 21.99968 | 23.08394 | 22.30749 | 20.68024 | 19.86071 | 25.14708 | 23.17134 |
| Tat₂ | R1 | 24.83638 | 24.98278 | 23.89314 | 23.05583 | 24.19563 | 23.67613 | 26.60516 | 26.46833 |
| | R2 | 25.98721 | 24.48807 | 24.27212 | 24.70116 | 24.78406 | 24.94172 | 27.22974 | 26.97839 |
| | R3 | 23.59364 | 22.32445 | 21.86751 | 21.99121 | 22.8027 | 22.26852 | 25.40135 | 24.36699 |
| Tat₂ + | R1 | 23.53142 | 24.62914 | 21.57527 | 22.68105 | 19.31091 | 21.34158 | 22.80724 | 25.29072 |
| TBPB | R2 | 24.90273 | 22.93241 | 23.09212 | 22.63462 | 24.23861 | 20.6065 | 25.99182 | 24.88507 |
| | R3 | 22.69374 | 21.69301 | 20.92079 | 21.20382 | 19.69844 | 22.30575 | 22.00518 | 18.81472 |
| siRNA | R1 | 23.6736 | 23.10535 | 22.51141 | 21.20382 | 22.75313 | 22.30575 | 25.13446 | 24.36139 |
| | R2 | 24.3346 | 24.86552 | 22.63000 | 22.78061 | 23.72576 | 23.80411 | 25.76133 | 26.07446 |
| | R3 | 25.12562 | 22.92009 | 22.77035 | 20.993 | 24.643 | 20.16918 | 26.48736 | 23.5526 |
| TBPB + | R1 | 23.95464 | 22.99029 | 21.96783 | 21.23991 | 19.47517 | 18.81622 | 23.77386 | 22.70514 |
| siRNA | R2 | 27.65538 | 23.44015 | 24.70874 | 22.83608 | 23.18448 | 20.70164 | 26.23117 | 25.17861 |
| | R3 | 22.77532 | 26.3533 | 21.36877 | 22.93817 | 18.8543 | 22.43513 | 23.76294 | 26.85483 |
| Tat₂ + | R1 | 23.69739 | 24.94946 | 22.79139 | 23.28964 | 23.56374 | 23.79088 | 26.56873 | 26.37328 |
| siRNA | R2 | 23.36801 | 22.79671 | 22.14104 | 22.68721 | 22.49702 | 23.12447 | 25.34292 | 25.88212 |
| | R3 | 26.69707 | 22.90538 | 23.55313 | 22.94949 | 24.65446 | 23.57826 | 26.85719 | 25.81883 |
| Tat₂ + | R1 | 25.5705 | 24.87389 | 22.25095 | 21.67845 | 21.568 | 19.85769 | 26.00412 | 24.39663 |
| TBPB + | R2 | 25.13404 | 24.65055 | 22.59306 | 23.53069 | 20.51949 | 21.75225 | 25.47359 | 26.23117 |
| siRNA | R3 | 25.40095 | 23.65055 | 23.52309 | 21.59999 | 21.22104 | 19.96935 | 26.25145 | 24.3767 |

| | | | | | | | | |
|---|---|---|---|---|---|---|---|---|
| General average | 24.65358 | 23.89809 | 22.76883 | 22.62324 | 22.29639 | 22.24792 | 25.48689 | 25.25128 |
| General Standard | 1.21450 | 1.31722 | 0.92368 | 0.95134 | 1.94878 | 1.81074 | 1.34342 | 1.95342 |
| Deviation | | | | | | | | |

¹MM = Maltose & Mannitol

adp-rf expression was found to be the most stable reference for normalization, with cdc and rl1 being far less stable. adp-rf had a standard deviation amongst all tested samples of less than 1 Ct, while all other reference genes displayed standard deviations of greater than 1.3 Ct. Additionally, it was found that cdc and rl1 samples that utilized TBPB had significantly lower Ct values than those without, with differences of up to 5 Ct, which represents an approximately 2⁵ fold difference in expression level. In the case of cdc this was a difference of 22-24 Ct to 19-22 Ct, and the case of rl1 a change from 24-26 Ct to 20-22 Ct and as low as 18 Ct. Therefore, adp-rf expression was used for normalization of the target pds gene expression level, based on the ΔΔCt method, as amplification efficiencies were substantially similar (within <2%). Data was displayed as relative expression of pds using 2^{-ΔΔCt} for conversion.

Statistical Analysis of RT-qPCR:

Data was analyzed using ANOVA and unpaired t-test to detect significant differences between pairs of means, with calibration of all treatments to an n=3 group of untreated controls. Repetitions that were furthest from the mean were removed from the data set to a minimum of four biological repetitions prior to ANOVA and t-test. This was done to maintain a balanced system for ANOVA. Statistical significance was rated at the 95% and 99.99% confidence interval, p<0.05 and p<0.0001 respectively. Two way ANOVA was applied twice to evaluate factors affecting relative expression of pds. The first application evaluated sample treatment (Control, TBPB, siRNA, Tat₂, Tat₂+siRNA, siRNA+TBPB, Tat₂+siRNA+TBPB, scsiRNA and Tat₂+scsiRNA+TBPB), and solvent used (ddH₂O or maltose-mannitol). The second application of two way ANOVA gauged possible interaction between sample treatments (Control, siRNA, Tat₂, Tat₂+siRNA and scsiRNA) with and without TBPB. In cases where ANOVA could not show statistical significance between factors, or interaction of factors, data sets were combined and reduced again to a minimum of eight biological replicates for display purposes and Student's t-test. Results of the analysis are shown in Table 4.

RT-qPCR of Inoculated Triticale Leaf Samples:

The expression level of the phytoene desaturase gene was evaluated using RT-qPCR, and converted to relative expression values using the 2^{-ΔΔCt} method. All data was calibrated against an untreated control (n=3). As seen in FIG. 5, reduction in expression by 20-30% was observed in all samples that were treated by injection. No statistical significance was found to be contributed by the solvent used (maltose-mannitol or ddH₂O) according to two-way ANOVA. A significance level of p=0.00024 was found for the difference between treatments containing TBPB and those without. Samples treated with TBPB showed up to 45% reduction in expression with Tat₂+TBPB, TBPB+siRNA and Tat₂+scsiRNA+TBPB showing significances of p<0.05. An approximately 75% reduction in pds expression (p<0.0001 compared to control) was only observed in treatments that possessed siRNA, TBPB and Tat₂. Additionally, only the Tat₂+siRNA+TBPB treatment was found to show a statistical difference from all other samples except for TBPB alone (p=0.068) in unpaired comparisons using Student's t-test. Furthermore, it must also be noted that an especially high degree of statistical significance was noted between the Tat₂+siRNA+TBPB treatment and the Tat₂+scsiRNA+TBPB treatment at p=4.21×10⁻⁴.

These results indicate that a composition containing TBPB in conjunction with Tat₂ and 21-base pair siRNA was most effective to induce silencing of the pds gene. It appears that the silencing may have been systemic, as the portion of leaf tissue sampled was at the tip, while injections were performed at the base of the leaf. It is thought that siRNA may have traveled via plasmadesmata or another vascular mechanism to induce silencing in the distally located tissue. In addition, no significant difference in the expression of pds was found between treatments using a maltose-mannitol solution as compared to ddH₂O, implying that siRNA nanocomplex tissue dispersion and cell entry was not significantly affected by the carrier used.

Without being bound by theory, it is considered that the significant down regulation of pds (up to 75-80%) by the Tat₂+siRNA+TBPB treatment may be due to the reduced size of nanocomplexes in that treatment. A size of between 10-20 nm is comparable to the size of pores in plant cell walls, which do not exceed 20 nm for most plants. It is believed that CPPs require contact with the plasma membrane to mediate import, and therefore smaller nanocomplexes and nanocomplexes having lower zeta-potentials may be imported into the cell and release the polynucleotide cargo more efficiently.

### Example 6: Delivery of siRNA in Colorado Potato Beetles

Colorado potato beetles (Leptinotarsa decemlineata) were obtained from Dr. Benoit Bizimungu's lab at Agriculture-Agri Food Canada, Fredericton, New Brunswick. Insects were maintained at 28° C. (16-h/8-h light/dark period) in plastic tubs and fed on potato foliage vegetative growth.

Multiple siRNA duplexes with two-nucleotide 3′ overhangs were designed to target mRNA transcribed from the p5cdh gene of L. decemlineata. The p5cdh gene codes for Δ¹-pyrroline-5-carboxylate dehydrogenase, which catalyses the oxidation of Δ¹-pyrroline-5-carboxylate to glutamate, the second step in the pathway by which proline is converted to glutamate to provide energy in the flight muscles of L. decemlineata. The siRNA duplexes were designed from the cDNA sequence of the p5cdh gene (GenBank Accession No. JN187429.2) using the siDirect calculator. The sequences of the siRNA duplexes are shown in Table 5.

TABLE 5
p5cdh siRNA sequences

| p5cdh | siRNA Sense | siRNA Antisense | Sense |
|---|---|---|---|
| siRNA | 5′-3′ | 5′-3′ | Position |
| 1 | AAUUUUGCUCGAUA | GUCAGUAUCGAGCAAAA | 521-541 |
| | CUGACUU | UUGA | |
| | (SEQ ID NO: 16) | (SEQ ID NO: 17) | |
| 2 | UGUAGAUAUGUAUG | GAGAACAUACAUAUCUA | 1015-1035 |
| | UUCUCUC | CAAG | |
| | (SEQ ID NO: 18) | (SEQ ID NO: 19) | |
| 3 | UUGCUUUUCUAAUC | CAGUAGAUUAGAAAAGC | 1804-1824 |
| | UACUGUU | AACA | |
| | (SEQ ID NO: 20) | (SEQ ID NO: 21) | |

The scrambled siRNA (scsiRNA) duplex of Example 5 (SEQ ID NO:6 and SEQ ID NO:7) was tested for potential off-target sequence alignment with genes in the insect databases, as well as the general NCBI database, using BLASTn. This scsiRNA was used as control to test for off-target effects of the test siRNA sequences. Chemically synthesized siRNA and scsiRNA sequences were dissolved in RNase-DNase free water (Ultrapure, Sigma) at a concentration of 1 mg/mL.

Nanocomplexes of the siRNA sequences were prepared in double distilled water (ddH₂O) at a ratio of 1:4:100 (siRNA:Tat₂:TBPB) by weight, which corresponds to a cationic:anionic (+/−) molar charge ratio of 1:4:100 as described in Example 2. 10 μL samples of increasing dosages of the nanocomplexes were administered to the mid-gut of insects using a calibrated syringe pump and a 30 gauge filed needle. Water alone was used as a control. The dosages used are shown in Table 6.

TABLE 6
Dosages of nanocomplexes administered to insects

| Treatment No. | siRNA (μg) | Tat₂ (μg) | TBPB (μg) |
|---|---|---|---|
| 0 (Control) | 0 | 0 | 0 |
| 1 | 0.5 | 2 | 50 |
| 2 | 1 | 4 | 100 |
| 3 | 1.5 | 6 | 150 |
| 4 | 2 | 8 | 200 |
| 5 | 2.5 | 10 | 250 |
| 6 | 3 | 12 | 300 |
| 7 | 3.5 | 14 | 350 |
| 8 | 4 | 16 | 400 |
| 9 | 4.5 | 18 | 450 |

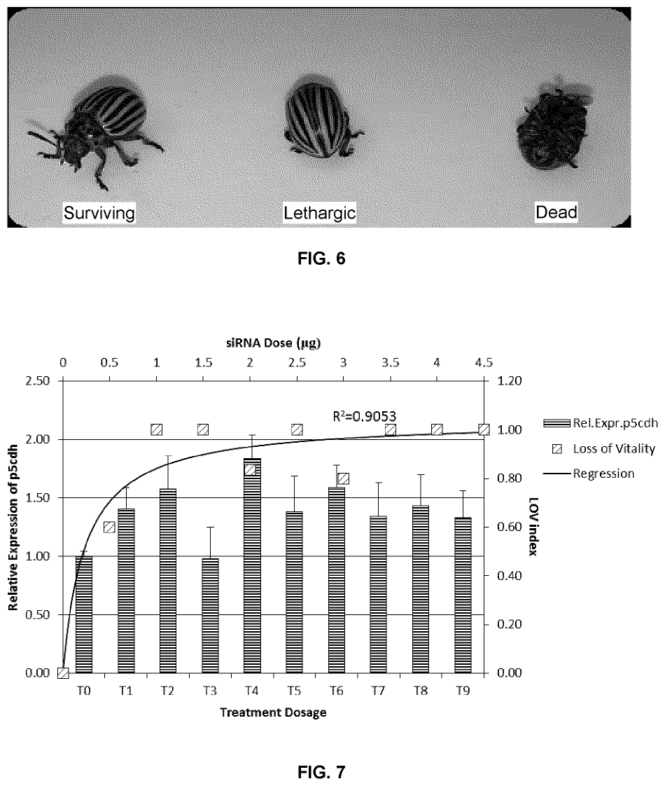

After administration of the treatments, the insects were transferred to labelled polyisopropylene tubs with black mesh over the lid with a square hole cut in the centre. The bottom of the tubs was lined with a sheet of moistened paper towel and potato leaves. The beetles were monitored for any phenotypic or behavioural changes and survivorship at 24 hrs post feeding. A fine brush was used to whisk the mouthparts of the beetles to check for movement, and numbers of surviving, lethargic and dead insects was scored. Insects typically scored as surviving, lethargic and dead, respectively, are shown from right to left in FIG. 6. Loss of vitality index (LoVI) was calculated using the equation

$$LoVI = 1 + \frac{( L - S )}{N}$$

where L is the number of lethargic insects 24 hours post feeding, S is the number of surviving insects 24 hours post feeding and N is the total number of insects fed. Death of insects was observed at 48 hours post feeding.

As seen in FIG. 7, mortality and lethargy were observed at doses of the siRNA nanocomplex containing as low as 0.5 μg of siRNA, and the LoVI increased with increasing dosage with good fit (R²=0.9053) to an hyperbolic curve.

RT-qPCR Analysis:

Three insects from each group treated with each dosage of siRNA nanocomplex were collected 24 hrs after feeding, flash frozen in tubes containing 6 stainless steel beads and ground down using a cryogrinder (Precellys™ 24; Bertin Technologies). RNA was extracted using an RNA extraction kit (RNeasy™ mini kit, Qiagen) DNA contamination was removed by DNase I (RNase free) treatment followed by cDNA synthesis using the SuperScript® VILO cDNA Synthesis Kit and Master Mix (Thermo Fisher). All RNA was validated for quality on denaturing bleach agarose gel. RT-qPCR was carried out under the conditions described in Example 5. Reference genes coding for ADP-ribosylation factor 1 (arf1) and the ribonuclease-like proteins ribosomal protein 4 (rp4) and ribosomal protein 18 (rp18) were used to normalize expression of the p5cdh gene. Quantification of relative mRNA level was performed based on the 2^{-ΔΔCt} method. The experimental replicates were individual insects (n=2-3 for all dosages). Primers used for the target and reference genes are shown in Table 7.

TABLE 7
Primer sequences

| | Forward primer | Reverse primer |
|---|---|---|
| Gene | (5′-3′) | (5′-3′) |
| p5cdh | TTGCATACACCCCAGCACTC | TTGACTACACCTGGCGGAAC |
| | (SEQ ID NO: 22) | (SEQ ID NO: 23) |
| arf1 | CGGTGCTGGTAAAACGACAA | TGACCTCCCAAATCCCAAAC |
| | (SEQ ID NO: 24) | (SEQ ID NO: 25) |
| rp4 | AAAGAAACGAGCATTGCCCTT | TTGTCGCTGACACTGTAGGGT |
| | CCG | TGA |
| | (SEQ ID NO: 26) | (SEQ ID NO: 27) |
| rp18 | TAGAATCCTCAAAGCAGGTGG | AGCTGGACCAAAGTGTTTCAC |
| | CGA | TGC |
| | (SEQ ID NO: 28) | (SEQ ID NO: 29) |

As seen in FIG. 7, treatment with p5cdh siRNA nanocomplexes at all dosages was associated with an increase in p5cdh expression, even though the treated insects experienced increased mortality.

A further experiment was carried out in which insects were fed either a dosage of p5cdh siRNA nanocomplexes prepared from 2.5 μg siRNA, 10 μg Tat₂, and 250 μg TBPB in water, or either water (control), 250 μg TBPB alone, 2.5 μg siRNA alone or a complex formed from 10 μg Tat₂ and 2.5 μg siRNA in the absence of TBPB. Insects were fed twice, at 24 hour intervals. At 24 hours after the second feeding, numbers of surviving, lethargic and dead insects was scored to determine LoVI, and expression of the reference gene arf1 and the target gene p5cdh was determined by RT-PCR as described above. Relative significances were determined by a Tukey-Kramer multiple comparisons test.

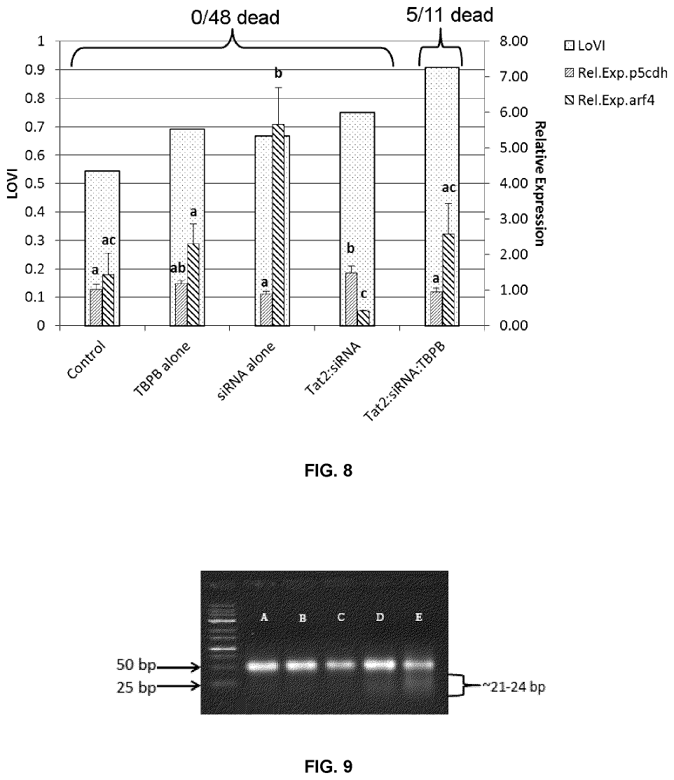

As seen in FIG. 8, the LoVI was significantly increased by treatment with p5cdh siRNA nanocomplexes compared to the other treatments. In addition, mortality was higher upon treatment with the nanocomplexes (5 dead of 11 insects treated compared to 0 dead out of 48 insects receiving the other treatments). However, the expression of p5cdh did not appear to be significantly decreased by treatment with p5cdh siRNA nanocomplexes compared to the other treatments. Without being bound by theory, it is thought that even though mRNA transcripts of the p5cdh gene may have been degraded by the presence of the p5cdh siRNA, expression of the p5cdh gene may have been increased by the insect cells to compensate. Indeed, as shown in FIG. 9, a greater abundance of small 21-24 bp RNA fragments were seen in lysates from insects treated with the nanocomplex (E) than in lysates from insects treated with water alone (A), TBPB alone (B), siRNA alone (C) or Tat₂-siRNA complexes in the absence of TBPB (D), indicating a higher degree of mRNA degradation.

Expression of the reference gene arf1 was unstable and had been noted to show a trigonometric relationship with the dosage of the p5cdh siRNA nanocomplexes. However, as seen in FIG. 8, expression of arf1 was stimulated to the greatest extent by the presence of siRNA alone, and to a much lesser extent by complexes with Tat₂, either in the presence or absence of TBPB. The expression product of this gene, ADP-ribosylation factor 1, is involved in endocytosis and intracellular trafficking, and it is thought that its expression may have been stimulated by the presence of the siRNA in the insect haemolymph.

The embodiments described herein are intended to be illustrative of the present compositions and methods and are not intended to limit the scope of the present invention. Various modifications and changes consistent with the description as a whole and which are readily apparent to the person of skill in the art are intended to be included. The appended claims should not be limited by the specific embodiments set forth in the examples, but should be given the broadest interpretation consistent with the description as a whole.

1. A composition for delivering a polynucleotide into a cell, the composition comprising a cell-penetrating peptide, the polynucleotide and a quaternary phosphonium salt.

2. The composition of claim 1 wherein the quaternary phosphonium salt is tetrabutylphosphonium bromide.

3. The composition of claim 1 wherein the polynucleotide is a double stranded RNA polynucleotide.

4. The composition of claim 3 wherein the double stranded RNA polynucleotide is a small interfering RNA (siRNA).

5. The composition of claim 1 wherein the molar charge ratio of the cell-penetrating peptide to the polynucleotide is between 1:1 and 8:1.

6. The composition of claim 1 wherein the molar charge ratio of the quaternary phosphonium salt to the polynucleotide is 100:1.

7. The composition of claim 1 wherein the cell is a plant cell.

8. A method of delivering a polynucleotide to a cell comprising exposing the cell to a composition according to claim 1.

9. A method of reducing or preventing expression of a gene in a cell, the method comprising exposing the cell to a composition comprising a cell-penetrating peptide, a double stranded RNA polynucleotide specific to the gene and a quaternary phosphonium salt.

10. The method of claim 9 wherein the quaternary phosphonium salt is tetrabutylphosphonium bromide.

11. The method of claim 9 wherein the molar charge ratio of the cell-penetrating peptide to the double stranded RNA polynucleotide is between 1:1 and 8:1.

12. The method of claim 9 wherein the molar charge ratio of the quaternary phosphonium salt to the double stranded RNA polynucleotide is 100:1.

13. The method of claim 9 wherein the double stranded RNA polynucleotide is a siRNA molecule having a sequence consisting of about 20 to about 30 nucleotides.


SEQUENCE LISTING (29 sequences)

SEQ ID NO: 1 - protein, 18 aa, Artificial Sequence
  Tat2
RKKRRQRRRRKKRRQRRR

SEQ ID NO: 2 - RNA, 18 nt, Artificial Sequence
  dsRNA 21mer
cauggagacgccgucguu

SEQ ID NO: 3 - RNA, 21 nt, Artificial Sequence
  dsRNA 21mer complimentary strand
cgaggacggcgucuccauguu

SEQ ID NO: 4 - RNA, 21 nt, Artificial Sequence
  PDS siRNA sense strand
cauguugugaagacacccgag

SEQ ID NO: 5 - RNA, 21 nt, Artificial Sequence
  PDS siRNA antisense strand
ucggugucuucacaacauggu

SEQ ID NO: 6 - DNA, 21 bp, Artificial Sequence
  scrambled PDS siRNA forward strand
agccgguacgaauagtgaguc

SEQ ID NO: 7 - RNA, 21 nt, Artificial Sequence
  scrambled PDS siRNA reverse strand
gacucacuauucguaccggcu

SEQ ID NO: 8 - DNA, 19 bp, Artificial Sequence
  PDS forward primer
aaagcagggtgttcctgat

SEQ ID NO: 9 - DNA, 23 bp, Artificial Sequence
  PDS reverse primer
catggataactcgtcagggttta

SEQ ID NO: 10 - DNA, 20 bp, Artificial Sequence
  ADP-RF forward primer
tctcatggttggtctcgatg

SEQ ID NO: 11 - DNA, 20 bp, Artificial Sequence
  ADP-RF reverse primer
ggatggtggtgacgatctct

SEQ ID NO: 12 - DNA, 20 bp, Artificial Sequence
  CDC forward primer
cagctgctgactgagatgga

SEQ ID NO: 13 - DNA, 20 bp, Artificial Sequence
  CDC reverse primer
atgtctggcctgttggtagc

SEQ ID NO: 14 - DNA, 20 bp, Artificial Sequence
  RL1 forward primer
ttgagcaactcatggaccag

SEQ ID NO: 15 - DNA, 20 bp, Artificial Sequence
  RL1 reverse primer
gctttccaaggcacaaacat

SEQ ID NO: 16 - RNA, 21 nt, Artificial Sequence
  p5cdh siRNA 521-541 sense
aauuuugcucgauacugacuu

SEQ ID NO: 17 - RNA, 21 nt, Artificial Sequence
  p5cdh siRNA 521-541 antisense
gucaguaucgagcaaaauuga

SEQ ID NO: 18 - RNA, 21 nt, Artificial Sequence
  p5cdh siRNA 1015-1035 sense
uguagauauguauguucucuc

SEQ ID NO: 19 - RNA, 21 nt, Artificial Sequence
  p5cdh siRNA 1015-1035 antisense
gagaacauacauaucuacaag

SEQ ID NO: 20 - RNA, 21 nt, Artificial Sequence
  p5cdh siRNA 1804-1824 sense
uugcuuuucuaaucuacuguu

SEQ ID NO: 21 - RNA, 21 nt, Artificial Sequence
  p5cdh siRNA 1804-1824 sense
caguagauuagaaaagcaaca

SEQ ID NO: 22 - DNA, 20 bp, Artificial Sequence
  p5cdh forward primer
ttgcatacaccccagcactc

SEQ ID NO: 23 - DNA, 20 bp, Artificial Sequence
  p5cdh reverse primer
ttgactacacctggcggaac

SEQ ID NO: 24 - DNA, 20 bp, Artificial Sequence
  arf1 forward primer
cggtgctggtaaaacgacaa

SEQ ID NO: 25 - DNA, 20 bp, Artificial Sequence
  arf1 reverse primer
tgacctcccaaatcccaaac

SEQ ID NO: 26 - DNA, 24 bp, Artificial Sequence
  rp4 forward primer
aaagaaacgagcattgcccttccg

SEQ ID NO: 27 - DNA, 24 bp, Artificial Sequence
  rp4 reverse primer
ttgtcgctgacactgtagggttga

SEQ ID NO: 28 - DNA, 24 bp, Artificial Sequence
  rp18 forward primer
tagaatcctcaaagcaggtggcga

SEQ ID NO: 29 - DNA, 24 bp, Artificial Sequence
  rp18 reverse primer
agctggaccaaagtgtttcactgc